(19) United States
(12) Patent Application Publication
(10) Pub. No.: US 2013/0245777 A1
(43) Pub. Date: Sep. 19, 2013

(54) KNEE SYSTEM

(71) Applicant: Gerald J. Jerry, Port Huron, MI (US)

(72) Inventor: Gerald J. Jerry, Port Huron, MI (US)

(21) Appl. No.: 13/881,610

(22) PCT Filed: Oct. 28, 2011
(86) PCT No.: PCT/US11/58291

§ 371 Date: Apr. 25, 2013

(30) Foreign Application Priority Data
Oct. 28, 2010 (US) 61407691

(51) Int. Cl. A61F 2/38

(52) U.S. Cl. CPC A61F 2/38
USPC 623/20.31; 623/20.21

(57) ABSTRACT

A knee system includes a knee prosthesis having a tibial arrangement including a tibial tray configured for attachment to a surgically-prepared surface of a proximal end of the tibia. The tibial arrangement also includes a tibial insert system including a lateral tibial insert and a medial tibial insert. One of the inserts is configured to move in a generally anterior-posterior direction relative to the tibial tray, and the other insert is configured for fixed attachment to the tibial tray. The knee prosthesis also includes a femoral component configured for attachment to a surgically-prepared surface of a distal end of a femur. The femoral component and tibial inserts each have articular surfaces configured to contact and articulate relative to each other during extension and flexion of the knee.

### CROSS-REFERENCE TO RELATED APPLICATIONS

This application claims the benefit of U.S. provisional patent application No. 61/407,691 filed 28 Oct. 2010, which is hereby incorporated herein by reference.

### TECHNICAL FIELD

The present invention relates to a surgically implantable knee system.

### BACKGROUND

Despite the advancements in implant design and surgical technique, total knee replacements still have certain limitations, and postoperative results can be less than desired. One issue that may arise is pain associated with movement of the knee, especially at deep flexion. There can be any number of causes for this, including inflammation of the soft tissue in and around the knee area. The inflammation may be caused by impingement of the tissue by the implant as it articulates, particularly when it articulates at the extremes of its range, such as during deep flexion. Therefore, it would be desirable to provide a knee replacement system that overcomes the problems described above, and allows a more anatomic range of motion for the patient without the pain associated with movements such as deep flexion.

### SUMMARY

Embodiments of the invention overcome at least some of the problems described above and provide a greater range of motion without the pain associated with some implant designs.

Embodiments of the invention include a knee system having separate medial and lateral tibial inserts. In at least some embodiments, the one of the medial or lateral insert is fixed, while the other insert is movable. Embodiments of the invention provide an anterior cruciate ligament (ACL) retaining design that allows for medial translation and roll back of the femoral component as it moves during flexion. If the tibial insert was a single-piece design, and was mobile on both sides—i.e., one full mobile unit rotating on a central axis—the lateral side could roll forward as the medial side translates posteriorly, causing more pressure to the lateral soft tissue envelope, which becomes in some instances inflamed and fibrotic leading to failure of implant satisfaction. Embodiments of the invention having a separate mobile insert and a separate fixed insert, help to avoid the problem of increased pressure to the lateral soft tissue envelope.

If both medial and lateral inserts are separate, and both are mobile, paradoxical anterior translation of the femoral component on tibia components could impinge the soft tissue envelope, causing repetitive trauma, bleeding, swelling, inflammation, scar contracture and possible failure. Embodiments of the invention having a separate mobile insert and a separate fixed insert, help to avoid this problem as well. In at least some embodiments, the tibial insert will be rounded, smooth and will have no greater a forward sagittal radius than the front of the tibial edge. This reduces or eliminates the soft tissue impingement. Also by fixing the one of the tibial inserts, bearing spit-out is inhibited.

Some embodiments include a circular undercut on the tibial tray that allows for a high interface contact and ease of obtaining a high polish. The surface of the tibial tray, which interfaces with the bottom of the tibial insert on the medial side, can either be flat or curvilinear in both the sagittal and coronal radii to control the bearing motion in the natural glide pattern. This can mimic the directional effect of the tibial spline to the femoral component. Also, a relief can be provided in the central concavity of the tibial tray increases the allowable thickness of the tibial insert.

Embodiments of the invention include a knee prosthesis for implantation in a knee. The knee prosthesis includes a tibial arrangement including a tibial tray configured for attachment to a surgically-prepared surface of a proximal end of a tibia, and a tibial insert system including a lateral tibial insert and a medial tibial insert. One of the lateral tibial insert or the medial tibial insert is a movable tibial insert configured to move in a generally anterior-posterior (A-P) direction relative to the tibial tray, and the other of the lateral tibial insert or the medial tibial insert is a fixed tibial insert configured for fixed attachment to the tibial tray. Each of the tibial inserts has a respective tibial articular surface. The knee prosthesis also includes a femoral component configured for attachment to a surgically-prepared surface of a distal end of a femur. The femoral component has a femoral articular surface. The femoral articular surface and the tibial articular surfaces are configured to contact each other and to articulate relative to each other during flexion and extension of the knee.

Embodiments of the invention also include a knee prosthesis for implantation in a knee having a tibial arrangement, which includes a tibial tray configured for attachment to a surgically-prepared surface of a proximal end of a tibia, and a tibial insert system configured for attachment to the tibial tray and including a tibial articular surface. The knee prosthesis also includes a femoral component configured for attachment to a surgically-prepared surface of a distal end of a femur. The femoral component includes a femoral articular surface configured to contact and articulate relative to the tibial articular surface during flexion and extension of the knee. The femoral component further includes a medial condyle and a lateral condyle narrower than the medial condyle for at least a portion of the condyles.

Embodiments of the invention further include a knee prosthesis for implantation in a knee. The knee prosthesis includes a tibial arrangement including a lateral tibial insert, a medial tibial insert, and a tibial tray configured for attachment to a surgically-prepared surface of a proximal end of a tibia. One of the lateral tibial insert or the medial tibial insert is a movable tibial insert configured to move relative to the tibial tray along an arcuate track in a generally anterior-posterior (A-P) direction. The other of the lateral tibial insert or the medial tibial insert is a fixed tibial insert configured for fixed attachment to the tibial tray. Each of the tibial inserts has a respective tibial bearing surface proximally disposed thereon. The knee prosthesis also includes a femoral component configured for attachment to a surgically-prepared surface of a distal end of a femur and having a femoral bearing surface configured to contact the tibial bearing surfaces.

### BRIEF DESCRIPTION OF THE DRAWINGS

FIG. 1 shows a knee prosthesis system in accordance with embodiments of the present invention;

FIG. 2 shows a top plan view of a lateral tibial insert in accordance with embodiments of the invention;

FIG. 3 shows a top plan view of a tibial tray in accordance with embodiments of the invention;

FIG. 4 shows a back view of the tibial tray shown in FIG. 3;

FIGS. 5A-5D show different views of a medial tibial insert in accordance with embodiments of the invention;

FIG. 6 shows a side view of a tibial tray in accordance with embodiments of the invention;

FIG. 7 shows an auxiliary view of the tibial tray shown in FIG. 6;

FIG. 8 shows a back view of a femoral component in accordance with embodiments of the invention;

FIG. 9 shows a top plan view of the knee system shown in FIG. 1;

FIG. 10 shows a cross-sectional view of a medial side of the knee system shown in FIG. 9; and

FIG. 11 shows a cross-sectional view of a lateral side of the knee system shown in FIG. 9.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

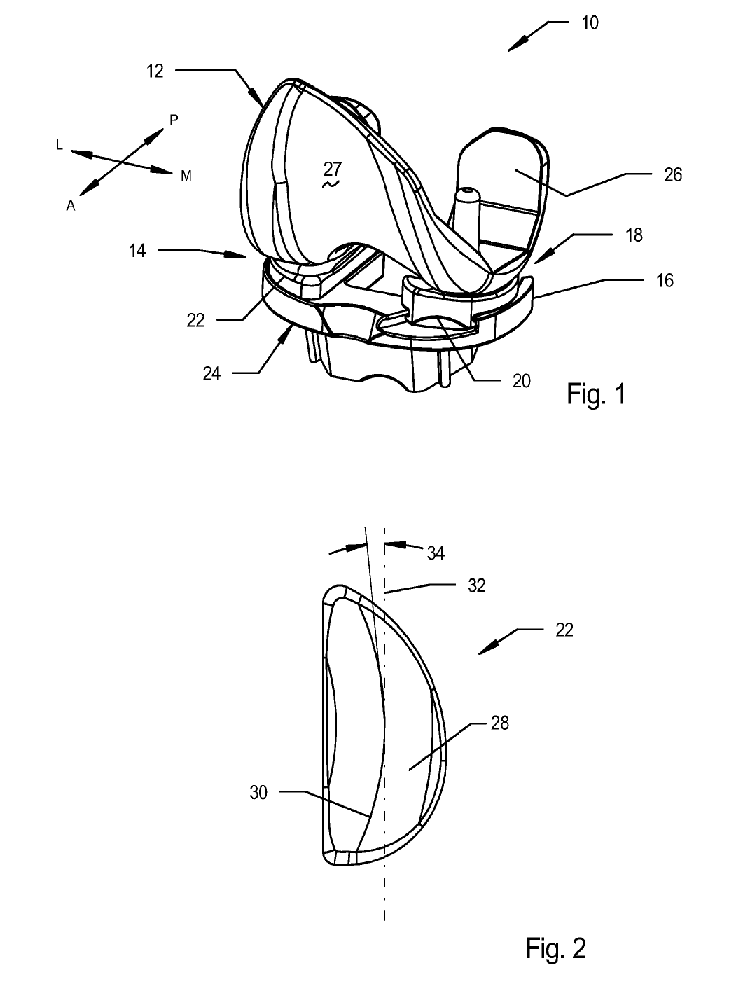

FIG. 1 shows a knee system 10 in accordance with embodiments of the present invention. The knee system 10 is a knee prosthesis configured for implantation in a mammalian knee. The knee prosthesis 10 includes a femoral component 12 and a tibial arrangement 14. The tibial arrangement includes a tibial tray 16 and a tibial insert system 18. In the embodiment shown in FIG. 1, the tibial insert system 18 includes separate medial and tibial inserts, 20, 22, with the medial tibial insert 20 being a movable tibial insert, and the lateral tibial insert 22 being a fixed tibial insert. As explained in more detail below, the medial tibial insert 20 is configured to move in a generally anterior-posterior direction relative to the tibial tray 16, and the lateral tibial insert 22 is configured for fixed attachment to the tibial tray 16. In other embodiments, a tibial insert system in accordance with the present invention may include separate medial and lateral inserts, each of which is fixed, or alternatively, a single tibial insert having medial and lateral portions integrated into a single unit.

The knee prosthesis 10 is a right knee, with the medial-lateral (M-L) and anterior-posterior (A-P) directions being indicated by the arrows shown in FIG. 1. The tibial tray 16 is configured for attachment to a surgically-prepared surface of a proximal and of the tibia, and in particular, a distal side 24 of the tibial tray 16 is configured to contact the prepared surface of the tibia. Similarly, the femoral component 12 is configured for attachment to a surgically-prepared surface of a distal end of a femur, and in particular, a proximal surface 26 of the femoral component 12 is configured to contact the prepared surface of the femur. Also shown in FIG. 1 is a patellofemoral groove 27, configured to provide a surface over which the patient's patella can articulate. As explained below, the patellofemoral groove 27 is configured such that the patella tracks more anatomically, which can result in a greater range of motion without pain.

FIG. 2 shows a top plan view of the lateral tibial insert 22. Lateral tibial insert 22 includes a tibial articular surface 28, which is configured to act as a bearing surface and to articulate relative to a femoral articular surface described below. As shown in FIG. 2, the articular surface 28 is generally arcuate, forming a curve 30 that moves medially at the A-P extremes, and moves laterally near the center of the insert 22. An A-P axis 32 shows that the arcuate curve 30 of the articular surface 28 is not symmetric, but rather, is offset by an angle 34. In the embodiment shown in FIG. 2, the angle 34 is approximately 2.5°; however, an angle between 2°-4° may be used, or even an angle between 1°-10°. Other angles, or indeed no offset angle, may be used depending on the desired geometric configuration.

As shown in FIG. 2, the geometry of the arcuate articular surface 28 helps to facilitate a natural movement of the knee during extension and flexion. To further facilitate normal anatomical function of the knee, the articular surface 28 of the tibial insert 22 may have a generally constant radius in a cross section taken through a sagittal plane—i.e., a cross section view in an M-L direction. Conversely, a cross section of the tibial insert 22 taken through a coronal plane—i.e., as viewed in an A-P direction—will show at least two different radii, a larger radius disposed toward the lateral edge of the tibial insert 22, and a smaller radius disposed toward the medial edge of the tibial insert 22. Notwithstanding the geometry of the embodiments shown in the drawing figures, embodiments of the present invention contemplate a lateral tibial articular surface of different radii in the sagittal cross section, and/or an articular surface with a single radius in the coronal cross section, depending on the desired geometry.

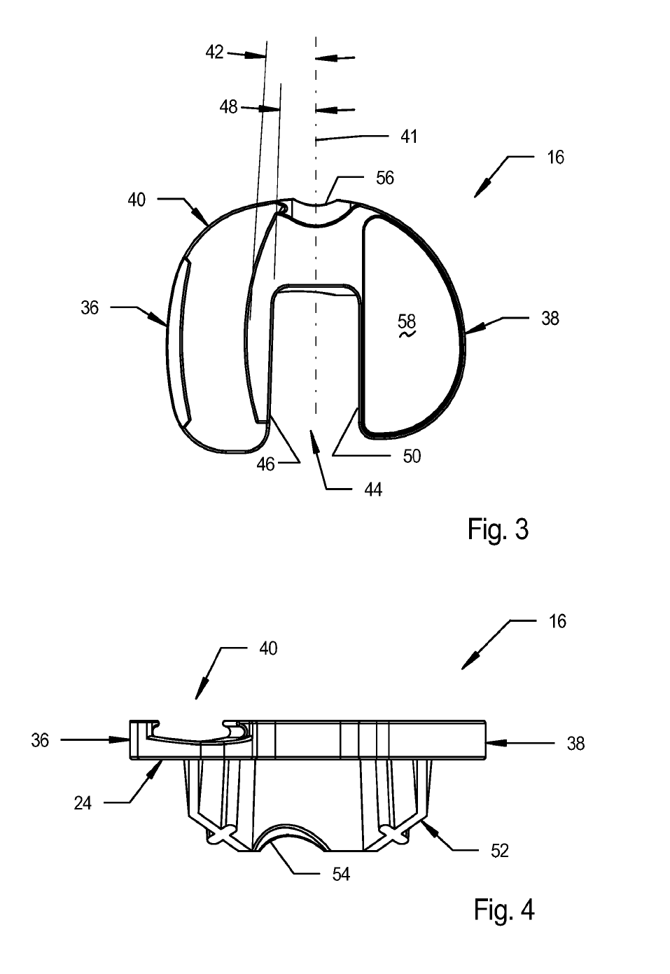

FIG. 3 shows a top plan view of the tibial tray 16. As discussed above, the tibial insert system 18 includes separate tibial inserts on the medial and lateral sides, with the medial tibial insert 20 being movable, and the lateral tibial insert 22 being fixed. To accommodate the inserts, the tibial tray 16 includes different geometric configurations on its medial side 36 and its lateral side 38. For example, the tibial tray 16 includes a track 40 disposed on the medial side 36, which is oriented in a generally A-P direction. As described in detail below, the movable, medial tibial insert 20 is configured to cooperate with the track 40 to allow movement of the medial tibial insert 20 in a generally A-P direction. As shown in FIG. 3, the track 40 is generally arcuate in shape, which provides a natural anatomical movement for the knee during extension and flexion.

Illustrated for reference in FIG. 3 is an A-P axis 41. As shown in FIG. 3, the arcuate track 40 is not symmetric relative to the medial side 36, but rather, is offset by an angle 42. In the embodiment shown in FIG. 3, the angle 42 is approximately 3°; however, an angle between 2°-4° may be used, or even an angle between 1°-10°. Other angles, or indeed no offset angle, may be used depending on the desired geometric configuration. Embodiments of the invention include offset arcuate paths for a track, such as the track 40, for an articular tibial surface, such as the articular surface 28 of the tibial insert 22—see FIG. 2—or for both. A patient may have a tibia that articulates along an offset arcuate path, with different patients having arcuate articulating paths offset by different angles. Indeed, the same patient may have different offset angles for each knee, and even different offset angles on the medial and lateral sides of the same knee. To facilitate a fit that is as close to anatomic as possible, radiographic information, for example, from a CT scan or MRI, can be used to choose a tray and insert combination having the right offset angles for each patient. In the embodiment shown in FIG. 3, the medial side 36 of the tibial tray 16 is longer in an A-P direction than the lateral side 38. Such a configuration lends itself well to having a movable tibial insert on the medial side 36, because the additional length provides more support for a movable insert that may overhang the anterior or posterior edge during extension and flexion of the knee.

The embodiment of the tibial tray 16 shown in FIG. 3, includes a notch 44 which acts as a cruciate cutout to accommodate the cruciate ligaments, and in fact, allows even the ACL to be preserved. To inhibit the likelihood of impingement of ligaments on the tray 16, the notch 44 opens slightly on its medial side 46 as it goes from anterior to posterior. In particular, the notch 44 includes a tapered medial side 46 which forms an angle 48 with A-P axis 41. In the embodiment shown in FIG. 3, the angle 48 is approximately 3°; however, an angle between 2°-4° may be used, or even an angle between 1°-10°. Other angles, or indeed no taper angle, may be used depending on the desired geometric configuration. In contrast to the medial side of 46, the notch 44 includes a lateral side 50 which is generally parallel to the A-P axis 41. Embodiments of the present invention may have a tray with a smaller notch, such that only the posterior cruciate ligament is retained, or it may have no notch, such that neither of the cruciate ligaments are retained.

FIG. 4 shows a back view of the tibial tray 16. In this view, a keel 52 is shown disposed on the distal side 24 of the tray 16. The keel 52 is configured for insertion into a tibia, and provides strength and support for a proximal portion of the tray 16. The keel 52 includes a recessed portion, or notch 54, which in this embodiment is generally arcuate in shape. The notch 54 provides an open area through which a replacement anterior cruciate ligament (ACL) can pass. It also provides room to pass a screw into the tibia after the implant is secured. This may be desirable, for example, in the event that a fracture appears in the tibia after the tray is cemented in place.

Returning to FIG. 3, it is shown that the tibial tray 16 includes a curved anterior surface 56 configured to provide an articular surface 40 patella, which may either be a patient's own patella, or a or a replacement or resurfaced patella making up part of a knee system such as the knee system 10. FIG. 3 also shows a lateral proximal portion 58 that is configured to receive the lateral tibial insert 22, which may attach to the tibial tray 16 via a snap fit or any other convenient method of attachment.

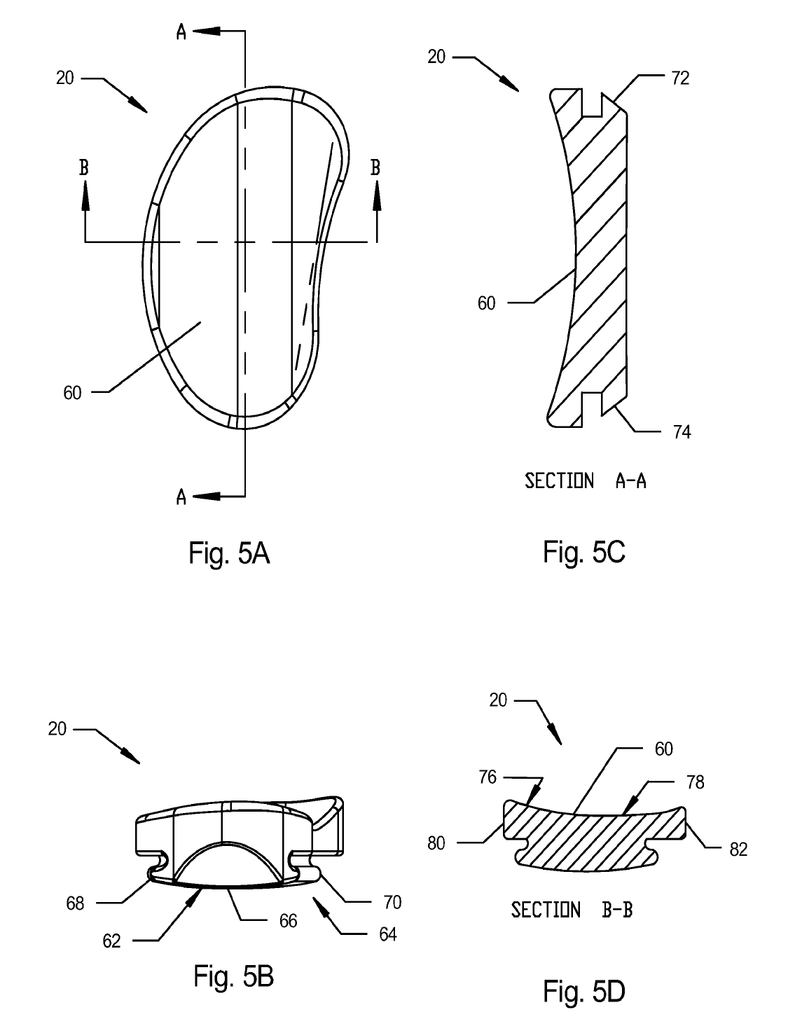

FIGS. 5A-5D show different views of the medial tibial insert 20. FIG. 5A shows a medial tibial articular surface 60, which is configured to act as a bearing surface and to articulate relative to a femoral articular surface described below. In the configuration shown in FIG. 5A, the articular surface 60 has a generally straight orientation in the A-P direction. The tibial insert 20 itself will, however, move in a generally arcuate path in the A-P direction as it articulates within the track 40 of the tibial tray 16.

As shown in FIG. 5B, the medial tibial inserts 20 includes a tracking feature 62, disposed on a distal side 64 of the insert 20. In the embodiment illustrated in FIG. 5B, the tracking feature 62 is a “key” configured to cooperate with the track 40—see FIG. 4—which is a “keyway”; this geometry may also be referred to as a “dovetail”. Tracking features and tracks other than keyways or dovetails are contemplated within the scope of the present invention. The tracking feature 62 has a thickness in a center portion 66 that is greater than the thickness of edge portions 68, 70. This is a result of the generally curved distal surface of the tracking feature 62, and helps to provide an overall greater thickness to the medial tibial insert in an area that would otherwise be thinner as a result of the articular surface 60.

This is further illustrated in FIG. 5C, which shows a sectional view of the medial tibial insert 20 taken through a sagittal plane. FIG. 5C also shows that the insert 20 includes chamfers 72, 74 respectively disposed at anterior and posterior ends, each of which helps to further reduce impingement on soft tissue at full extension and deep flexion. FIG. 5D shows another sectional view of the medial tibial insert 20 taken through a coronal plane. As shown in FIG. 5D, the articular surface 60 includes at least two different radii 76, 78 as viewed in the coronal section. The first radius 76 is smaller, and provides a steeper slope toward a medial side 80 of the tibial insert 20. Conversely, the radius 78 is larger than the radius 76, which provides a more gradual and reduced slope toward a lateral side 82 of the insert 20.

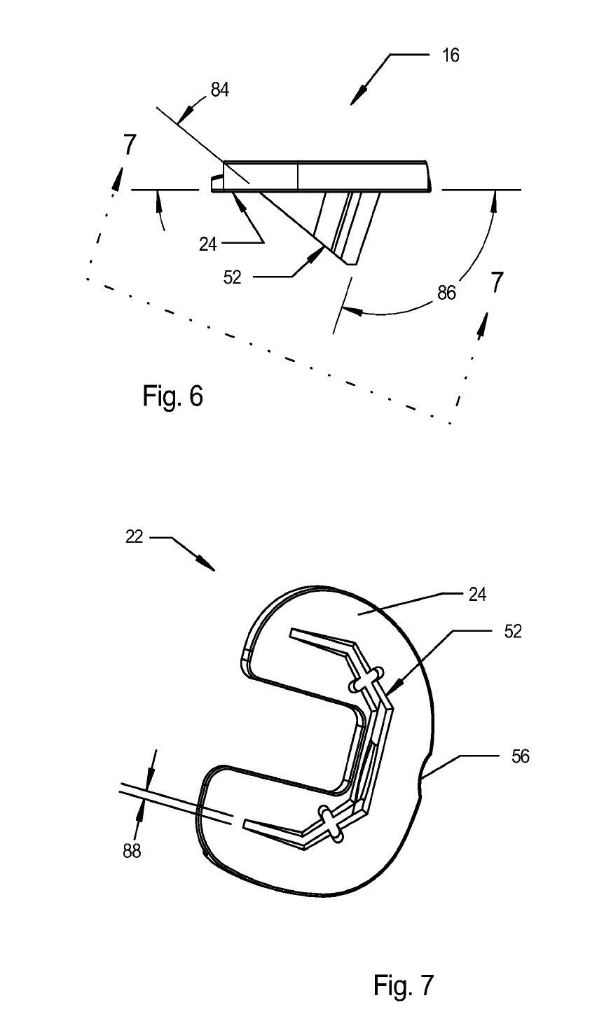

FIG. 6 shows a side view of the tibial tray 16, and in particular, shows the angular geometry of the keel 52. The keel 52 forms an angle 84 with a surface on the distal side 24 of the tray 16. In the embodiment shown in FIG. 6, angle 84 is approximately 37°; however, different angles may be used. Toward the anterior side of the tray 16, the keel 52 forms an angle 86 with a surface of the distal side 24 of approximately 110°. Again, this angle may change for different sizes of implants, or even for the same sized implant in different embodiments of the invention. FIG. 7 shows the distal side 24 of the tray 22. In this view, a thickness 88 of one portion of the keel 52 is shown, which in this embodiment is approximately 3 mm.

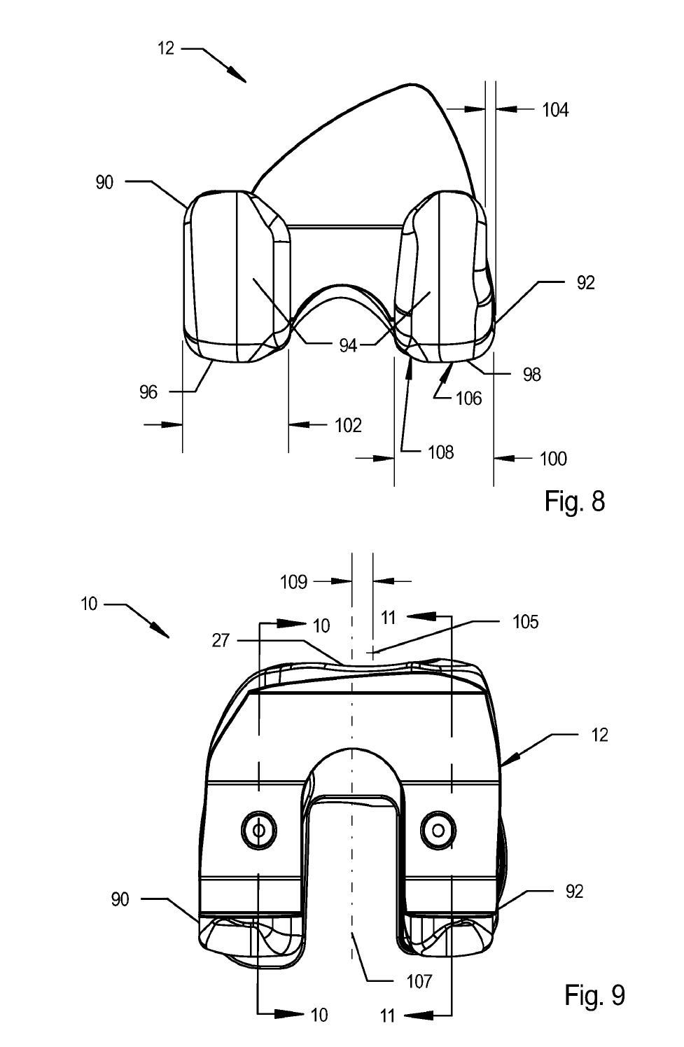

FIG. 8 shows a back view of the femoral component 12 of the knee system 10. The femoral component 12 includes a medial condyle 90 and a lateral condyle 92. The femoral component 12 also includes a femoral articular surface 94, which may be conveniently divided into a medial femoral articular surface 96 and a lateral femoral articular surface 98. The medial and lateral femoral articular surfaces 96, 98 are configured to respectively contact the articular surface 60 of the medial tibial insert 20 and the articular surface 28 of the lateral tibial insert 22, such that they articulate relative to each other during extension and flexion of the knee.

As shown in FIG. 8, a width 100 of the lateral condyle 92 is less than a width 102 of the medial condyle 90. In at least one embodiment, the width of the medial condyle is approximately 25 mm and the width of the lateral condyle is approximately 23.5 mm. This difference in width may be present for an entire articulating surface length of the condyles 90, 92, or it may be present only for a portion of the articulating surface, particularly near a posterior end of the condyles 90, 92. The posterior ends of the condyles will contact their respective tibial insert articulating surfaces at deep flexion. The difference in condylar width is another feature of embodiments of the present invention that helps to facilitate deep flexion of the knee without soft tissue impingement and the pain associated with it. Another feature of the femoral component 12 illustrated in FIG. 8 is an offset, or recess 104, near the posterior portion of the lateral side of the lateral condyle 92. The recess 104 is another feature of embodiments of the present invention that helps to facilitate deep flexion of the knee without soft tissue impingement, and in particular, the recess 104 helps to avoid impingement with the popliteal tendon.

As oriented in FIG. 8, the medial and lateral articular surfaces 96, 98 appear as lines in a coronal plane. These lines, and thus their respective articular surfaces 96, 98, are made up of one or more radii to better articulate with their respective tibial articular surfaces 60, 28. In the embodiment shown in FIG. 8, the lateral femoral articular surface 98 includes two separate radii 106, 108. The first radius 106 covers approximately 75% of the lateral femoral articular surface 98, and is disposed toward the lateral side. The second radius 108 is smaller than the first radius 106, and covers about 25% of the lateral femoral articular surface 98, and it is disposed toward the medial side.

Values of the radii 106, 108 may vary depending on a number of factors, including the geometric configuration of the cooperating articular surfaces of the tibial insert or inserts. Providing a smaller radius near the medial side of the lateral condyle 92 helps to reduce soft tissue impingement, particularly at deep flexion. Even so, a “radius”, such as the radius 108 may be increased significantly until it essentially becomes a straight line or chamfer, though for purposes of description herein, may still be referred to as a “radius”. In general, the second “radius” is configured to reduce the amount of material toward the medial side of the lateral articular surface 98, which, as noted above, helps reduce soft tissue impingement.

FIG. 9 shows a top plan view of the knee system 10, and in particular the femoral component 12. A point 105 represents a center of the patellofemoral groove 27, and as shown in FIG. 9 it is offset from a central axis 107 of the femoral component 12. The central axis 107 represents a center line between the outer edges of the medial and lateral condyles 90, 92. In the embodiment shown in FIG. 9, the offset distance 109 is approximately 1-2 mm, although other offset distances may be used depending on the desired line of tracking for the patient's patella. Having the center of the patellofemoral groove 27 offset toward the lateral side works in concert with the narrower lateral condyle 92, the combination of which helps provide a more anatomic tracking for the patient's patella. The can reduce stress on the patellar tendon and in at least some cases allow the patient's own patella to be resurfaced without the need for a patellar implant.

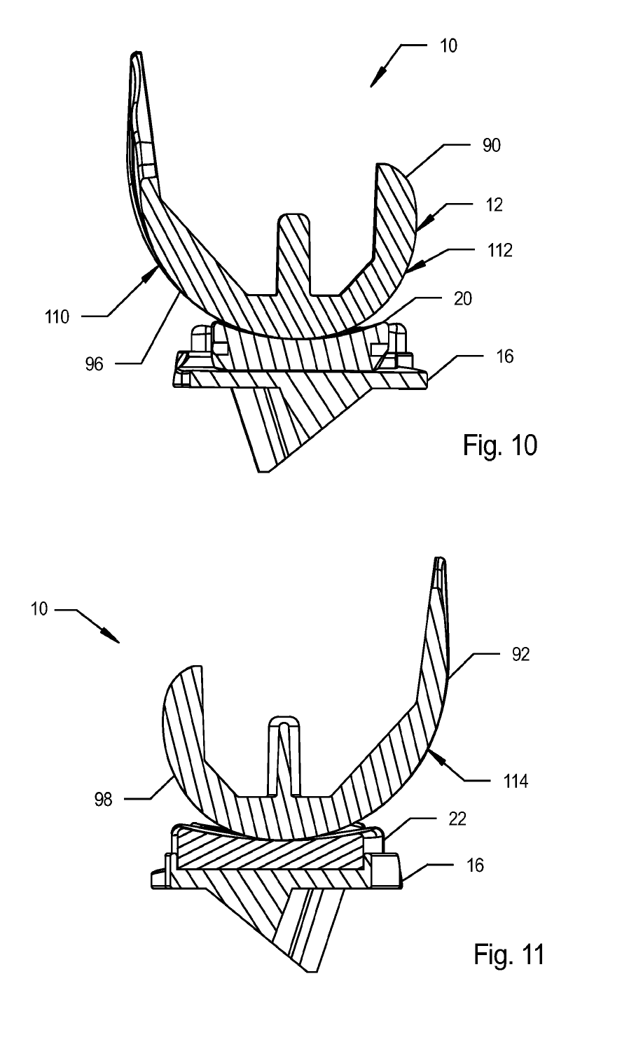

In FIG. 9, section lines 10-10 and 11-11 show the orientations for FIGS. 10 and 11, respectively. FIG. 10 shows a side sectional view of the knee system 10, and in particular, a section taken through the medial condyle 90 of the femoral component 12, the medial tibial insert 20, and the medial side 36 of the tibial tray 16. The sectional view of the medial condyle 90, is shown in FIG. 10 in a sagittal plane view. In the embodiment shown in FIG. 10, the articular surface 96 of the medial condyle 90 has two distinct radii 110, 112. The radius 110 is generally constant, and sweeps over approximately 120° of the articular surface 96. Near the posterior side of the articular surface 96, the radius changes and becomes much smaller to further accommodate deep flexion of the knee.

FIG. 11 shows a sectional view of the lateral condyle 92, also through a sagittal plane. In this view, the lateral femoral articular surface 98 is shown as a curvilinear line 114. In the embodiment shown in FIG. 11, the line 114 includes a plurality of radii which are specifically chosen to mimic the natural curvature of the articulating surface of the lateral condyle of an anatomic knee. Both the articulating surface 98 of the lateral femoral condyle 92 and the articulating surface 96 of the medial femoral condyle 90 may have a different radius or radii than those illustrated in the embodiments shown in the drawing figures. The specific selection of articulating surface geometry may depend on a number of factors, including whether one or more of the tibial inserts is movable as discussed above, what the configuration of the mating articular surface of the tibial insert or inserts is, and whether the tibial insert system is one piece or has separate medial and lateral components.

The components described in the knee system 10, and the components of other embodiments of the present invention, may be made from any material having the engineering properties and physiologic compatibility desired for such an implant. For example, various metals, such as cobalt chrome and titanium alloys, can be used. One or more of the surfaces of these metal components can be porous coated or covered with hydroxyapatite, or other materials known to facilitate bone growth. Ceramic materials may also be used, as well as polymeric materials, particularly for a tibial insert system. It should also be noted that as used herein, a “sagittal” plane need not bisect the body into two equal halves; rather, it refers to a plane cuts anywhere through the body in an anterior-posterior direction. Therefore sections viewed in a sagittal plane are viewed in a medial-lateral direction. Similarly, as used herein, a “coronal” plane need not bisect the body into two equal halves; rather, it refers to a plane cuts anywhere through the body in a medial-lateral direction. Therefore sections viewed in a coronal plane are viewed in an anterior-posterior direction.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A knee prosthesis for implantation in a knee, comprising:
a tibial arrangement including a tibial tray configured for attachment to a surgically-prepared surface of a proximal end of a tibia, and a tibial insert system including a lateral tibial insert and a medial tibial insert, one of the lateral tibial insert or the medial tibial insert being a movable tibial insert configured to move in a generally anterior-posterior (A-P) direction relative to the tibial tray and the other of the lateral tibial insert or the medial tibial insert being a fixed tibial insert configured for fixed attachment to the tibial tray, each of the tibial inserts having a respective tibial articular surface; and
a femoral component configured for attachment to a surgically-prepared surface of a distal end of a femur and having a femoral articular surface, the femoral articular surface and the tibial articular surfaces being configured to contact each other and to articulate relative to each other during flexion and extension of the knee.

2. The knee prosthesis of claim 1, wherein the tibial tray includes a track on one of the medial side or the lateral side of the tibial tray, the track being oriented in a generally A-P direction, the movable tibial insert including a tracking feature disposed on a side opposite the articular surface of the movable tibial insert, the tracking feature being configured to cooperate with the track to allow the movable tibial insert to move in a generally A-P direction during extension and flexion of the knee.

3. The knee prosthesis of claim 2, wherein the tracking feature has thickness in a center portion that is greater than thicknesses at edge portions of the tracking feature when viewed in a coronal plane.

4. The knee prosthesis of claim 2, wherein the track is generally arcuate and is offset from an A-P axis between about one degree and ten degrees.

5. The knee prosthesis of claim 2 wherein the articular surface of the fixed insert is generally arcuate and is offset from an A-P axis between about one degree and ten degrees.

6. The knee prosthesis of claim 1, wherein the tibial tray includes a keel disposed on a distal side thereof, the keel including a recessed portion configured to accommodate a replacement anterior cruciate ligament.

7. The knee prosthesis of claim 1, wherein the tibial tray includes a cruciate cutout disposed between the medial and lateral sides thereof, the cruciate cutout having a lateral side generally parallel to an A-P axis and a medial side offset from the A-P axis between about one and ten degrees.

8. The knee prosthesis of claim 1, wherein the tibial tray includes a curved anterior surface configured to provide an articular surface for a patella.

9. The knee prosthesis of claim 1, wherein the femoral component includes a medial condyle and a lateral condyle, the lateral condyle being narrower than the medial condyle for at least a portion of the condyles.

10. The knee prosthesis of claim 9, wherein the lateral condyle includes a recess disposed along a lateral side of the posterior portion thereof.

11. The knee prosthesis of claim 1, wherein the femoral articular surface includes a medial articular surface and a lateral articular surface, the lateral articular surface defining first and second radii as viewed in a coronal plane, the first radius being disposed toward the lateral side of the lateral articular surface and over more than half of the lateral articular surface, the second radius being different from the first radius, and configured to reduce an amount of material of the lateral articular surface toward the medial side of the lateral articular surface.

12. A knee prosthesis for implantation in a knee, comprising:
a tibial arrangement including a tibial tray configured for attachment to a surgically-prepared surface of a proximal end of a tibia, and a tibial insert system configured for attachment to the tibial tray and including a tibial articular surface; and
a femoral component configured for attachment to a surgically-prepared surface of a distal end of a femur and including a femoral articular surface configured to contact and articulate relative to the tibial articular surface during flexion and extension of the knee, the femoral component further including a medial condyle and a lateral condyle narrower than the medial condyle for at least a portion of the condyles.

13. The knee prosthesis of claim 12, wherein the lateral condyle includes a recess disposed along a lateral side of the posterior portion thereof.

14. The knee prosthesis of claim 12, wherein the femoral articular surface includes a medial articular surface and a lateral articular surface, the lateral articular surface defining first and second radii as viewed in a coronal plane, the first radius being disposed toward the lateral side of the lateral articular surface and over more than half of the lateral articular surface, the second radius being different from the first radius, and configured to reduce an amount of material of the lateral articular surface toward the medial side of the lateral articular surface.

15. The knee prosthesis of claim 12, wherein the femoral component defines a central axis and further includes a patellofemoral groove configured to provide an articular surface for a patella, the patellofemoral groove having a center laterally offset from the central axis.

16. The knee prosthesis of claim 12, wherein the tibial insert system includes a medial tibial insert configured to move in a generally anterior-posterior (A-P) direction relative to the tibial tray, and a lateral tibial insert separate from the medial tibial insert and configured for fixed attachment to the tibial tray.

17. The knee prosthesis of claim 16, wherein the tibial tray includes a track on a medial side of the tibial tray, the track being oriented in a generally A-P direction, the medial tibial insert including a tracking feature disposed on a side opposite the articular surface of the medial tibial insert, the tracking feature being configured to cooperate with the track to allow the medial tibial insert to move in a generally A-P direction during extension and flexion of the knee.

18. The knee prosthesis of claim 17, wherein the track is generally arcuate and is offset from an A-P axis between about one degree and ten degrees.

19. A knee prosthesis for implantation in a knee, comprising:
a tibial arrangement including a lateral tibial insert, a medial tibial insert, and a tibial tray configured for attachment to a surgically-prepared surface of a proximal end of a tibia, one of the lateral tibial insert or the medial tibial insert being a movable tibial insert configured to move relative to the tibial tray along an arcuate track in a generally anterior-posterior (A-P) direction and the other of the lateral tibial insert or the medial tibial insert being a fixed tibial insert configured for fixed attachment to the tibial tray, each of the tibial inserts having a respective tibial bearing surface proximally disposed thereon; and
a femoral component configured for attachment to a surgically-prepared surface of a distal end of a femur and having a femoral bearing surface configured to contact the tibial bearing surfaces.

20. The knee prosthesis of claim 19, wherein the medial tibial insert is the movable tibial insert, and the fixed tibial insert is the lateral tibial insert, the medial tibial insert including a key and the tibial tray including a keyway disposed on the medial side thereof and configured to receive the key to facilitate movement of the medial insert along the arcuate track.

21. The knee prosthesis of claim 20, wherein the keyway is generally arcuate and is offset from an A-P axis between about one degree and ten degrees.

22. The knee prosthesis of claim 21, wherein the tibial bearing surface of the lateral tibial insert is generally arcuate and is offset from an A-P axis between about one degree and ten degrees.

23. The knee prosthesis of claim 22, wherein the offset for at least one of the keyway and the tibial bearing surface of the lateral tibial insert is chosen based on radiographic information specific to a particular patient.
